(19) United States
(12) Patent Application Publication
(10) Pub. No.: US 2021/0346545 A1
(43) Pub. Date: Nov. 11, 2021

(54) APPARATUS FOR STERILIZING THE INTERIOR OF VEHICLES, ETC. AND METHODS OF USE

(71) Applicant: Samantha Ashley Carlin, 1055 Squires Drive, PA (US)

(72) Inventor: Samantha Ashley Carlin, 1055 Squires Drive, PA (US)

(21) Appl. No.: 17/241,055

(22) Filed: Apr. 26, 2021

Related U.S. Application Data
(60) Provisional application No. 63/015,647, filed on Apr. 26, 2020.

(51) Int. Cl. A61L 2/10; A61L 2/26; A61L 9/20

(52) U.S. Cl. CPC A61L 2/10; A61L 2/26; A61L 9/20; A61L 2209/12; A61L 2202/16; A61L 2202/25; A61L 2202/14

(57) ABSTRACT

A system including a sterilization device, an optional mobile application and a method of use for disinfecting the interior of automobiles, working or living spaces. Each sterilization device includes at least one Ultraviolet-C (UV-C) light source, which attaches to the inside cabin of a car using Velcro or any suitable attachment means. Each location where a UV-C light is installed may include a plurality of UV-C lights that are positioned to emit light in particular directions to sterilize different areas of the car. Each device may include one or more motors used to rotate the UV-C lights for maximum coverage of the space, as well as a circuit board to communicate with a remote control or mobile application for remote use and monitoring of the sterilization device.

### CROSS-REFERENCE TO RELATED APPLICATIONS

This utility application claims the benefit under 35 U.S. Code § 119(e) Provisional Application Ser. No. 63/015,647 filed on Apr. 26, 2020, entitled Apparatus and Method for Sterilizing the Interior of a Vehicle. The entire disclosure of the provisional application is incorporated by reference herein.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISK

Not Applicable

### FIELD OF THE INVENTION

The disclosed invention relates to a sterilization device and more particularly to a sterilization device using Ultraviolet-C (UV-C) germicidal light to disinfect the interior of vehicles and interior spaces, etc. to minimize the chance that any pathogen found in the space will not infect the residing individuals.

### BACKGROUND OF THE INVENTION

With the recent outbreak of the Coronavirus disease 2019 (COVID-19), people have serious trepidation about traveling inside a rideshare automobile, such as a taxi, limousine, and rideshare-service car, in which this virus and other microbes, viruses, and pathogens can be deposited by passengers entering and exiting the cabin of the automobile.

For instance, there is concern that passengers will deposit Coronavirus or other pathogens on the seatbelts, car seats, door handles, floor, air inside the cabin, and other areas inside an automobile, which it may not be possible to effectively eliminate with spray disinfectants, nor practical in between dropping off one passenger to a destination, and then picking up a new passenger for another trip.

What is needed, therefore, is an improved system of disinfecting the interior of an automobile compartment; especially after a prior passenger exits the vehicle, but before a new passenger enters the vehicle, i.e., in between “fares” such as in association with rideshare services or taxis. The system should be easy to install and use.

This same system can also be applied to the interior of office spaces, household rooms and living spaces, to clean these spaces before and after use.

### SUMMARY OF THE INVENTION

The following summary describes features of the various embodiments of the invention. It is not intended to limit the description of the invention in any way.

The disclosed improvements comprise a system and method including an optional mobile application that satisfies the need to disinfect the inside of the cabin of a vehicle or interior space.

In a first aspect, a system for disinfecting the interior of the automobile is provided, the system comprises: at least one UV-C light source that attaches to the inside compartment of the car, such as the car ceiling using Velcro or any suitable attachment means. For instance, the UV-C light source may be attached in a central area of the ceiling of a car using Velcro, clips, or other suitable mechanisms.

In a second aspect, the UV-C light may attach to the lip of a sunroof (if the car is equipped with a sunroof) or even the counsel of the front of the car.

In a third aspect, a system for disinfecting the interior of the automobile may include multiple UV-C light sources, such as two or more devices with a primary and replica relation, attached to the back of one or more headrests.

As appreciated by those skilled in the art after having the benefit of this disclosure, each location in which a UV-C light source is installed may include a plurality of UV-C lights that are positioned to emit light in particular targeted directions to sterilize different areas of the compartment of the car for maximal coverage.

Still further, in a fourth aspect, a motor may be attached to one or more of the UV-C light sources to rotate the light(s) to shine in different directions for maximum coverage. For example, the UV-C light(s) may be rotated up and down 180 degrees to cover the floor and ceiling of the vehicle, or other areas in which the light is directed as it rotates from 0 to 180 degrees. In other embodiments, UV-C light may be directed to only specific areas of the vehicle, such as the rear compartment of the vehicle.

In a fifth aspect, a system for disinfecting the interior of the automobile may include at least one or more UV-C lights positioned inside a spherical-shell, which protects one or more UV-C light units contained within the shell from accidental jostling by a passenger and for covering lighting mechanisms for aesthetic purposes. As described above, the light(s) positioned within the shell may rotate by motorized means and/or may remain fixed in different implementations. In one embodiment, the UV-lights in the shell may be positioned or rotated to bathe large swaths of the interior compartment of the vehicle in UV-C light for deep cleaning of the vehicle's interior.

In a sixth aspect, the system may include motion sensors positioned inside the car or attached to the device, for detecting the motion of people or animals inside the car, or whether the compartment is empty. In one embodiment, motion sensor(s) may be positioned to detect motion in specific areas of the vehicle, such as the rear seats, the front-passenger seat, and/or the front seats, etc. The motion sensors used in conjunction with a control system may prevent the lighting system to activate when people or animals are detected in the vehicle, or in certain targeted areas of the vehicle, such as the rear seats.

In a seventh aspect, a communication interface, or circuit board, configured to communicate with a mobile computing device containing a processor, and/or computer app operating in conjunction with the mobile-computing device, are configured to control and to activate the UV-C light(s) to sanitize one or more areas of the compartment of the vehicle.

In an eighth aspect, a system as detailed above is provided, wherein there is at least one mobile computing device which includes a display configured to provide a representation of different modes of operation of the UV-C light systems, such as to direct light to bathe the rear seats of the vehicle only, such as when a driver of a taxi or rideshare service is in between fares and desires to continue to remain in the driver's seat while the UV-C light(s) clean only the rear-interior portion of the vehicle.

In a ninth aspect, a system as detailed above is provided wherein the display is further configured to provide further modes of operation for a user to select, which send instructions to the UV-C light(s) to bathe the entire interior compartment of the vehicle, and for specified duration of time to ensure the vehicle's interior is substantially sterilized. For instance, if there are no humans or humans in the vehicle, the user may select a mode of operation that illuminates the entire interior compartment of the vehicle for a sufficient period of time, such as 60 minutes, to kill pathogens that may have built up over time in the vehicle. Different modes of operation may be selected by the user to activate automatically, such as when no humans or animals are detected in the vehicle by the motion detectors. Or the user may manually select when to activate and deactivate the UV-C light systems, including targeting only specific areas of the vehicle via an app.

In another aspect, a mobile application is provided that, for example, includes an interface allowing users to select modes of operation of the control system for the UV-C lights to target areas of a vehicle for cleaning (i.e., UV illumination), either automatically or manually.

This summary is provided to introduce a selection of concepts in a simplified form that are further described below. This summary is not necessarily intended to identify key features or essential features of the claimed subject matter, nor is it necessarily intended to be used as an aid in determining the scope of the claimed subject matter.

Although a number of direct-to-consumer and household UV-C products exist, there are no UV-C products on the market that are specifically designed to clean the car interior, such that they come with the necessary hardware for mounting and placement.

Moreover, there are no UV-C lighting solutions that account for situations such as ridesharing and the need for rapid on-the-go cleaning.

Additionally, there are no UV-C lights that are specifically designed to allow the user to be in the car while the UV-C light is on.

Lastly, although there are some UV-C lights that come with a remote control, there are no UV-C products on the market that have a compatible mobile application to control the light.

As used herein, an automobile and car, are meant to refer generally to any type of vehicle that is driven on roads, including cars, trucks, and related vehicles, and may be broadened to cover interior compartments of other vehicles including, busses, trains, and even airplanes.

Reference herein to “example,” “embodiments” or similar formulations means that a particular feature, structure, operation or characteristic described in connection with the example, is included in at least one implementation in this description. Thus, the appearance of such phrases or formulations herein are not necessarily all referring to the same example. Further, various particular features, structures, operations, or characteristics may be combined in any suitable manner in or more examples.

### BRIEF DESCRIPTION OF THE DRAWING FIGURES

FIG. 1 is an isometric view of one exemplary embodiment of the device depicted in a hemisphere implementation whereby the hemisphere contains a dome-light or half-spherical-shaped housing containing UV-C lights. The flat surface of the hemisphere attaches to the interior car ceiling or walls, directing the UV-C light outward through the dome.

FIG. 2 is a side elevation view of a second embodiment of the device depicting an upside-down hexagonal pyramid-like structure, in which UV-C light-emitting diodes (LEDs) are positioned on each of the six sides of the pyramid to provide ample coverage of the area being disinfected. In this embodiment, the device attaches to the ceiling of the car cabin via the flat pyramid base. Additionally, in this embodiment, a motion sensor is depicted as a dome attached to the bottom of the device, positioned downward to detect motion below.

FIG. 3 is the bottom elevation view of FIG. 2 showing the placement of the motion sensor depicted as a circle within a rectangle, as well as the placement of an additional UV-C light located on the bottom face of the hexagonal pyramid designed to disinfect the area directly below. Additionally, a small standalone circle is pictured on the bottom of the device depicting the emergency off button.

FIG. 4 is an isometric view taken from a side oblique angle of a rectangular embodiment of the sterilization device depicting a rectangular shell which houses a strip of UV-C LEDs. In this depiction, the strip of UV-C LEDs are angled downward showing the ability of the light strip to rotate up and down via a motor to provide maximum coverage from floor to ceiling of the area being disinfected. In this view, a charging port and an emergency off button are depicted on the side of the rectangular housing.

FIG. 5 is a front elevation view of the embodiment of the device shown in FIG. 4, but with the UV-C LED strip rotated inward in the OFF position.

FIG. 6 is a bottom elevation view of the embodiment shown in FIG. 4, depicting a second emergency off switch located on the bottom of the device.

FIG. 7 is a back elevation view of the embodiment shown in FIG. 4, depicting the ventilation slots for the interior control board and hardware to prevent the device from over-heating. Additionally, the two sets of four vertical lines depicted on the back of the device serve as the female slots by which an optional headrest attachment can attach to the device.

FIG. 8 is an isometric view taken from the side oblique angle of the adjustable headrest attachment used to attach the rectangular embodiment of the sterilization device pictured in FIG. 4 to the back of the car headrests. The rectangular clip pictured in the foreground connects to the female slots of the sterilization device, and the adjustable spring-loaded tube pictured in the background connects the device to the metal prongs of the headrest.

FIG. 9 is a front view of the home screen of a mobile application used to control the sterilization device referred to as The Purple Bug on the mobile application screen. The mobile application features the ability to turn on and off the sterilization device by pressing the clean button displayed on the home screen.

FIG. 10 is a front view of the scheduling feature available in the mobile application depicted in FIG. 9. Within the scheduler, the user of the device can select the day (Sunday to Saturday) and time for the sterilization device to activate the cleaning mode.

### DETAILED DESCRIPTION OF THE PREFERRED EMBODIMENTS

One embodiment of a system according to the present invention is a system for a UV-C germicidal light that is used to disinfect air, surfaces, and crevices within the interior of an automobile.

UV-C, also known as ultraviolet C, is ultraviolet light with wavelengths between 200 and 290 nanometers. UV-C light works to disinfect the interior of a car by killing or inactivating microorganisms such as bacteria and viruses that the light comes in contact with. The UV-C light kills these microorganisms by denaturing their nucleic acids and disrupting their DNA so that they can no longer function.

Traditionally, the use of UV-C light includes the disinfection of hospitals, water purification facilities, nursing homes, workstations, schools and equipment. Additionally, considering the Coronavirus outbreak, UV-C has become one of the many technologies being utilized to help reduce and control the spread of the virus.

Placement & Design:

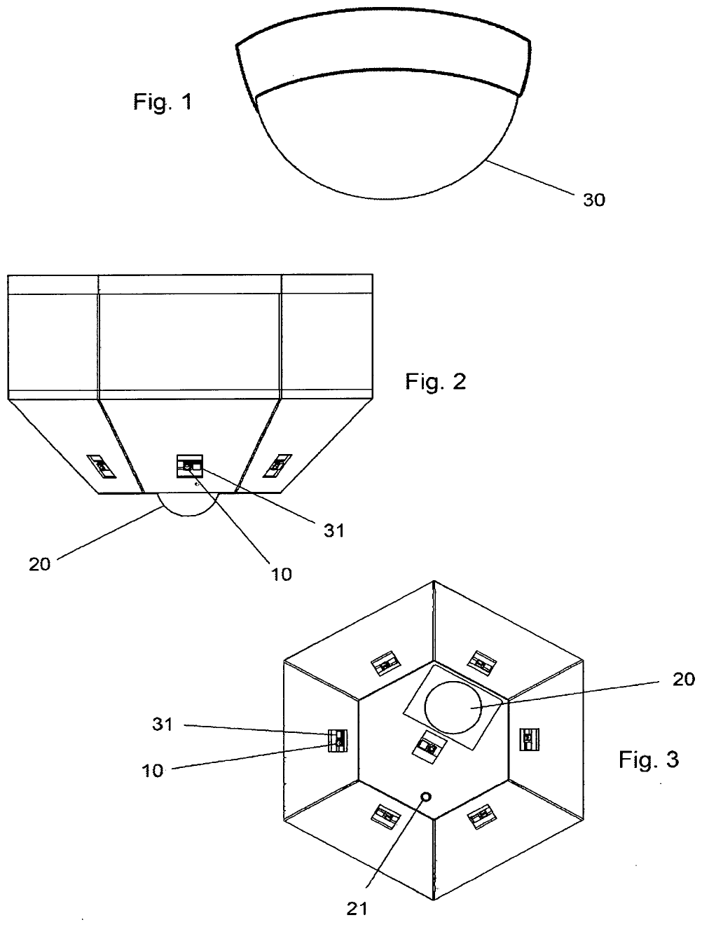

In this first embodiment, a circular UV-C light in the shape of a hemisphere (See FIG. 1) is installed inside a vehicle and attaches to the ceiling of the car. The hemisphere includes a dome-light or half-spherical-shaped housing 30 for containing UV-C light(s) therein, power supply, wiring, motor(s) (optional), and a means for attaching the housing to the ceiling of a car. As appreciated by those skilled in the art after having the benefit of this disclosure, the housing may be implemented in other shapes such as cubic, pyramid (See FIG. 2), or various other shapes.

The hemisphere can be attached via Velcro or via metal (or plastic) clips that attach to the lip of the sunroof opening.

In one aspect, the housing may be placed directly above the center console in order to provide ample coverage of both the front-seat and the back-seat area. As the UV-C light only disinfects surfaces that the light comes into contact with, placing the light on the ceiling provides the most coverage, if only a single housing is used.

Moreover, there can be more than one UV-C light in the shell in order to increase output. In one aspect, the UV-light(s) are mounted vertically in the shell 30, perpendicular to the ceiling. In another aspect the UV-C light(s) can sit horizontally in the shell 30, parallel to the car ceiling. In a third aspect as depicted in FIG. 2, the UV-C lights in the form of UV-C light-emitting diodes 10, commonly referred to as UV-C LEDs, are positioned along the perimeter of the device. In this embodiment, the shell is a multi-sided pyramid, with each face featuring a window or small cut out allowing the UV-C LEDs to shine through.

Additionally, there can also be a motor attached to the UV-C lights allowing the lights to rotate within shell encasement, during cleaning, i.e., when the UV-C lights are activated.

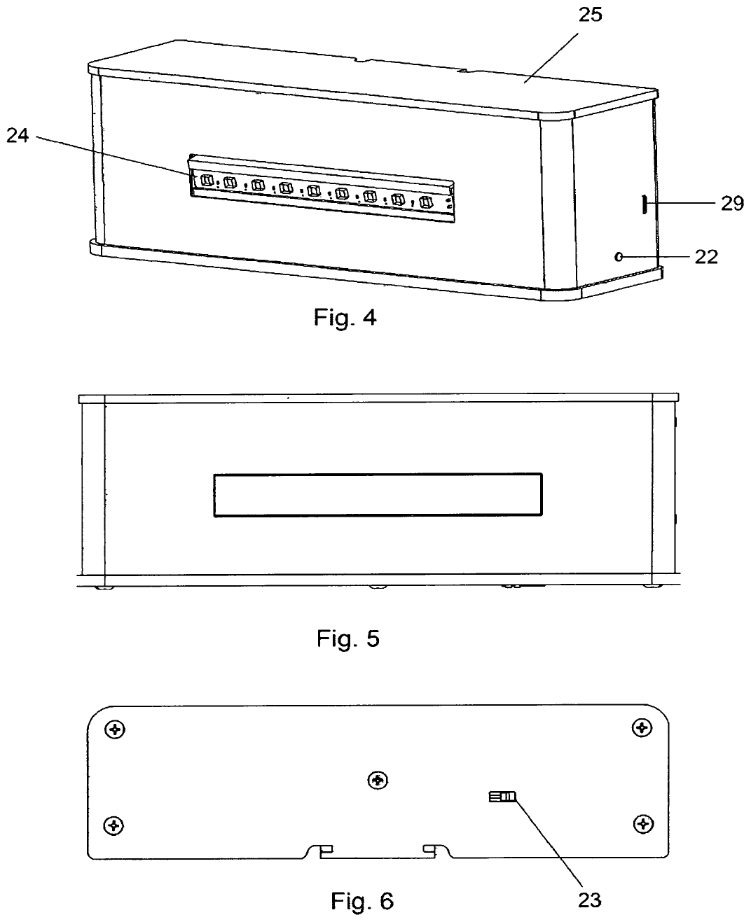

Ina second embodiment of the design, the system may include a rectangular collection of lights 24, within a rectangular encasement 25 such as shown in FIG. 4 with lighting 24 directed in the down direction. In this aspect, the lighting system provides targeted disinfection of the back-passenger area and attaches to the back of the driver and passenger seats. This design is primarily for taxi drivers or rideshare drivers who continually have passengers in and out of the back seat of the car.

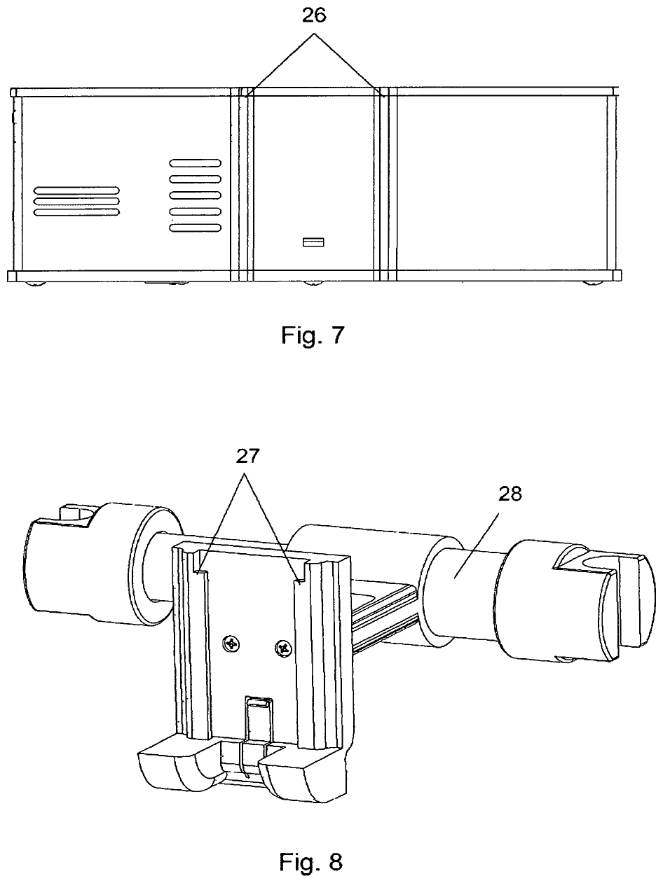

In this embodiment, the plurality of lights attach to the back of the driver and passenger seats via an adjustable headrest assembly (See FIG. 8), which allows the lighting system to securely attach to the metal prongs of the headrest. The back of the rectangular encasement (See FIG. 7) contains slots 26 which serve as the female component, which attaches to the male component 27 of the headrest assembly to secure the device to the assembly. The headrest assembly is then attached to the prongs of the headrest, using a spring-loaded tube 28.

The design also includes a built-in motor to rotate the light from floor to ceiling during cleaning.

Functionality:

To activate the lighting system, the user may use an application operating on their smart phone or computer system that communicates with the lighting system.

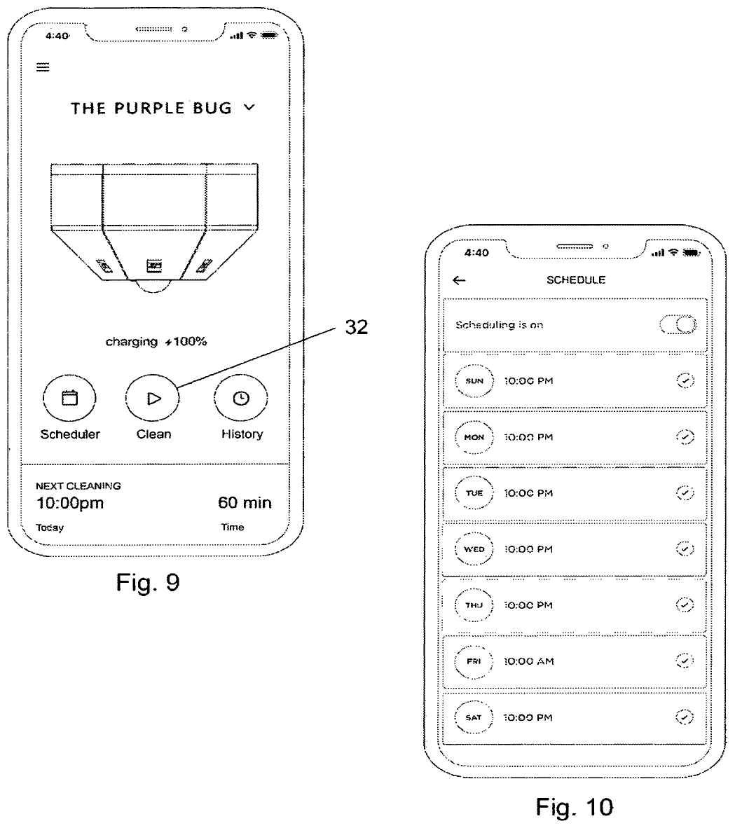

For instance, the user may select “The Purple Bug” mobile application as pictured in FIG. 9, and depress the clean button 32. In the hemisphere embodiment, the UV-C light turns on for a period of approximately 60 minutes, disinfecting all areas where the light hits. Additionally, in the hemisphere embodiment, all passengers and animals should be out of the vehicle as direct exposure to the UV-C rays can be harmful.

In the rectangular embodiment for ride sharing, the device turns on for a period of 1-2 minutes. In this embodiment, a motor is attached to each UV-C light enabling the light to rotate up to 180 degrees from floor to ceiling during the cleaning. Moreover, in the rectangular embodiment, the driver is able to remain in the front seat of the car as the light is directed at the back seat only.

Timing:

The time required to disinfect the car is directly correlated to the strength of UV-C light and the total surface area being cleaned. In the hemisphere embodiment, the targeted cleaning time is approximately 60 minutes to account for the full interior of the car. On the other hand, in the rectangular embodiment, the targeted clean time is 1-2 minutes. To achieve these cleaning times, suitable UV-C strength lights can be used in to achieve desired strength of UV-C light.

Charging:

In one embodiment, the lighting system is able to charge via a 12-volt outlet, which is the voltage of the cigarette lighter in a car. In this this embodiment, the lighting system is plugged into the cigarette lighter or an additional 12V outlet available in some car models. This allows for the lighting system to charge whenever the vehicle is turned on providing a convenient and hassle-free charging experience.

In another aspect, the system can be plugged directly into a standard household outlet of 120V using a converter for inputs of 100-120V and outputs of 12V.

In a third aspect, the lighting system does not include a rechargeable battery, and the battery must be replaced after a specified period of time.

Safety Features:

The lighting system may include a built-in motion sensor(s) 20 as shown in FIG. 2 and FIG. 3 which will prevent the lighting system from turning on at its scheduled time if there is movement in the car, and/or it will turn off the lighting system during its cleaning if motion is detected. As direct exposure to UV-C light can potentially be dangerous to humans, as well as animals, it is important that the design have these safety measures in place in the event that someone is in the car while the UV-C light is on.

Moreover, there is no manual “ON” button for the device in order to prevent accidental exposure. Instead, the user can only activate the device by using the compatible mobile application or separate remote-control device.

In the rectangular embodiment of the lighting system, for taxi and rideshare drivers, the UV-C light can only be turned on by the driver when the driver hits the “clean” button via an app or via another remote device. Specifically, in this embodiment of the design, cleanings cannot be scheduled. As passengers will be getting in and out of the vehicle at different times throughout the day, a scheduled cleaning could result in a passenger being exposed to the UV-C light.

In both the spherical and rectangular embodiments of the design, there is an emergency off button 21 as seen in FIG. 3 and an emergency off button 22 as seen in FIG. 4, in the event that the passenger or driver cannot access their cell phone to control the light.

Specifically, in the rectangular embodiment seen in FIG. 4, there is an emergency off button 22 located on the side of the device, as well as an emergency off switch 23 located on the bottom of the device. The emergency off button 22 will tell the circuit board to turn off UV-C lights and to stop the cleaning cycle, whereas the emergency sliding switch 23, when turned off, will prevent the current from flowing to the UV-C lights by breaking the circuit.

Additional safety features for the rectangular embodiment of the lighting system for taxi and rideshare drivers, include the motorized turning of the light(s) inward away from the exterior of the device as shown in FIG. 5, such that if the UV-C light(s) were to unintentionally turn on, the UV-C light(s) would not face the occupants in the backseat area.

Next, as UV-C light is colorless, the device will also include colored lighting such as purple lights or purple light-emitting diodes (LEDs) 31, along with the UV-C light(s) 10, in order to visually indicate when device is turned on.

Lastly, as UV-C light does not transmit through materials, humans or animals passing by a vehicle undergoing a cleaning session will not be harmed as long as the vehicle's windows and doors are properly closed.

Example Mobile App:

Using a mobile application as depicted in FIG. 9, the user can remotely manage the UV-C lighting system. Via the app, the user can monitor the cleaning, as well as start, stop, or pause the cleaning by depressing the clean button 32, view a history of past cleanings, view the charge level of the battery, and schedule the car for cleanings as in the first embodiment of the design. The scheduling feature depicted in FIG. 10, is applicable to the everyday consumer who wishes to schedule their car for daily or weekly cleanings at times when the car is parked and no passengers are in the vehicle.

### CONCLUSION

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

I claim:

1. An electronic device for sterilizing the interior of vehicles or interior spaces using Ultraviolet-C (UV-C) light such that viruses and bacteria found in the air and on surfaces that come in contact with the UV-C light will be destroyed by said device, said device comprising:
a. a hard material made of plastic, glass, or a blend of materials serving as the encasement of the device with specified open areas to emit the UV-C light to targeted areas;
b. a set of UV-C light(s) placed inside of the encasement, emitting light outward through the specified open areas of the encasement;
c. a set of colored lights placed inside the encasement, alongside or next to the UV-C light, positioned to emit light outward through the specified open areas of the encasement;
d. a circuit board placed inside of the encasement allowing for the remote control of the device using Wi-Fi or Bluetooth capabilities accessed via a compatible mobile application or a separate remote control device;
e. a rechargeable battery connected to the circuit board placed inside of the encasement to provide power to the device;
f. a standard 12V cigarette lighter power cord for recharging the battery in the vehicle;
g. a wall charger for inputs of 100-120V and outputs of 12V for recharging the battery in a home, office, or interior setting;
h. a stepper motor and compatible driver connected to the circuit board inside of the encasement to provide rotation of the UV-C light(s);
i. an emergency off button(s) used to manually turn off the device;
j. a passive infrared sensor (PIR sensor) to detect motion in the immediate surrounding area of the device up to 10 meters (30 feet), such that if motion is detected in the car or interior space, the circuit board will stop the cleaning cycle or will not begin the cleaning cycle.

2. The encasement of claim 1, wherein the said encasement is shaped as a hemisphere, such that the flat side of the hemisphere can be attached to the ceiling of a car, living space, etc.

3. The encasement of claim 2, such that the flat side of the hemisphere encasement is made of plastic or metal, and the circular portion of the encasement is made of clear plastic or glass, allowing the light to be emitted through the translucent circular portion of the hemisphere encasement.

4. The encasement of claim 2, such that open areas, windows or holes are made in the circular portion of the hemisphere encasement, allowing light to emit through the designated open areas.

5. The encasement of claim 1, wherein the said encasement of the design is a multi-faced pyramid with a flat surface at the vertex rather than a point, allowing for the placement of a motion sensor and additional lighting on the surface, which is parallel to the base of greater surface area attached to the car ceiling.

6. The encasement of claim 5, such that the base connects to the ceiling or flat surface, and the faces of the pyramid allow for the transmission of light through designated open areas or holes by which the UV-C lights will be internally attached to emit light outwards.

7. In the encasement of claim 5, such that the base connects to the ceiling or flat surface, and the faces of the pyramid will be made of a translucent plastic or glass allowing for the emission of light from the Ultraviolet UV-C lights placed within.

8. The system of claim 1, wherein the battery has a designated lifetime and is not rechargeable.

9. The system of claim 1, wherein both the UV-C lights and colored lights can be in the form of bulbs.

10. The system of claim 1, wherein the UV-C lights and colored lights can be in the form of UV-C light emitting diodes (also known as UV-C LEDs).

11. A lighting system for sterilizing surfaces and air using UV-C light such that viruses and bacteria found in the interior of rideshare vehicles such as taxis, limousines, and rideshare-service cars, will be destroyed by said device, said device comprising:
a. a primary and replica device, such that the primary device controls the replica device and serves as the communication hub;
b. separate rectangular encasements for each device made of hard plastic, glass, or a blend of materials with specified open areas to emit the UV-C light in specific directions;
c. a set of interior UV-C lights placed inside each encasement, emitting light outward through the specified open areas of the encasement;
d. a set of colored lights placed inside each encasement, alongside or next to the UV-C light, positioned to emit light outward through the specified open areas of the encasement;
e. two headrest attachments, one for the primary device and one for the replica device, that allow the devices to securely, but not permanently, attach to the back of the driver and passenger seat headrests, wherein the open area of the encasement is faced toward the backseat passenger area;
f. a circuit board placed inside of the primary encasement allowing for the remote control of the device using Wi-Fi or Bluetooth capabilities accessed via a compatible mobile application or a separate remote control device;
g. a rechargeable battery placed inside the primary device to provide power to both the primary and replica devices.
h. a standard 12V cigarette lighter power cord for recharging the battery in a vehicle;
i. a wall charger for inputs of 100-120V and outputs of 12V for recharging the battery in a home, office, or interior setting;
j. a connection cord between the primary and replica device allowing for the primary device to control the replica;
k. a stepper motor and compatible stepper motor driver installed in both the primary and replica devices used to rotate the UV-C lights to provide ample coverage of the backseat passenger area from floor to ceiling, approximately 180 degrees;
l. an emergency off button, located on the primary device, used to communicate with the circuit board to turn off the UVC-lights and stop the cleaning cycle;
m. an emergency sliding switch, located on the primary device, used to prevent the current from flowing to the UVC-lights by breaking the circuit.

12. The system of claim 11, wherein the two devices do not have a primary and replica relation, such that each device can be controlled independently through the use of the mobile application or remote control device, this said system comprises:
a. one circuit board for each device;
b. an emergency off button on each device;
c. an emergency off switch on each device.

13. The system of claim 11, wherein one device, including all the components of the primary device, serves to disinfect the entire backseat area of the vehicle.

14. The system of claim 11, wherein both the UV-C lights and colored lights can be in the form of bulbs.

15. The system of claim 11, wherein the UV-C lights and colored lights can be in the form of UV-C light emitting diodes (also known as UV-C LEDs).
